(19) United States
(12) Patent Application Publication
(10) Pub. No.: US 2024/0299173 A1
(43) Pub. Date: Sep. 12, 2024

(54) SACROILIAC JOINT FIXATION DEVICE WITH ADJUSTABLE CONNECTOR ASSEMBLY

(71) Applicant: Phoenyx Spinal Technologies, Inc., Tallahasee, FL (US)

(72) Inventor: Bret Michael Berry, Tallahassee, FL (US)

(21) Appl. No.: 18/180,337

(22) Filed: Mar. 8, 2023

(51) Int. Cl. A61F 2/30

(52) U.S. Cl. CPC A61F 2/30; A61F 2002/30507; A61F 2002/30537

(57) ABSTRACT

The present invention is generally directed to an implant for sacroiliac (SI) Joint fixation. Specifically, the present invention is generally directed to an implant for SI Joint fixation comprising a pair of anchor members connected to one another by an adjustable connector assembly. In accordance with embodiments of the present invention, the dual anchor pieces may be inserted into the sacroiliac joint, a first anchor member into the sacrum, a second anchor member into the ilium. An adjustable connector assembly may hold the pair of anchor members together. The adjustable connector assembly may be used to distract or compress the sacroiliac joint.

### FIELD OF THE INVENTION

The present invention is generally directed to an implant for sacroiliac (SI) Joint fixation. Specifically, the present invention is generally directed to an implant for SI Joint fixation comprising a pair of anchor members connected to one another by an adjustable connector assembly.

### BACKGROUND

Sacroiliac joints may be affected by arthritis, traumatic injury, pregnancy, infection, and other conditions, including systemic inflammatory conditions. Movement of such sacroiliac joints can be a major source of pain. In some scenarios, fixating the sacroiliac joint has been shown to alleviate such pain. Traditionally, dowels or wedges made from cadaveric bone have been used as an implant to help fixate the joint. In such instances, the implant is configured to be inserted from an inferior-posterior approach, and generally hammered into a space between the sacrum and ilium.

More recently, catamaran styled implants have been developed, for example, by companies such as Tenon Medical (https://tenonmed.com/catamaran/, hereinafter the “Tenon Medical Implant”). The Tenon Medical Implant is configured to stretch across an SI Joint in order to fixate it. However, the Tenon Medical Implant is a single, monolithic piece. Such a configuration may limit the ability to manipulate the anatomy of pelvic bones or sacroiliac joints and/or make revisions or adjustments to same.

The present invention is configured to provide an adjustable implant configured, for example, to distract or compress a sacroiliac joint. In some examples, a dual anchor assembly may be inserted into the sacroiliac joint, a first anchor member into the sacrum, a second anchor member into the ilium, with an adjustable connector assembly holding the pair of anchor members together and enabling the anchors to separate to distract a sacroiliac joint. These and other features and advantages of the present invention will be explained and will become obvious to one skilled in the art through the summary of the invention that follows.

### SUMMARY OF THE INVENTION

The present invention relates generally to an SI joint implant for at least partial to full fixation SI joints. In particular, the present invention encompasses an implant with a pair of anchor members and an adjustable connector adapted to support fixation of an SI joint.

It is an object of the present invention to provide an implant configured to be inserted into a sacral-iliac joint.

It is another object of the present invention to provide an SI joint implant for partial to full fixation of SI joints.

It is another object of the present invention to provide an SI joint implant with an integrated adjustable connector assembly.

It is another object of the present invention to provide an SI joint implant with arm members slidably integrated within anchor member shafts.

It is another object of the present invention to provide an SI joint implant comprising a first anchor member configured to be implanted into the sacrum, and a second anchor member configured to be implanted contralateral of the SI Joint, into the ilium with respect to the first anchor member.

It is another object of the present invention to provide an SI joint implant having a pair of anchor members configured to be relatively close to one another during insertion (e.g., a collapsed state), and, to be expanded once the implant is suitably positioned in the SI joint.

It is another object of the present invention to provide an SI joint implant configured to transfix SI joints.

It is another object of the present invention to provide an SI joint implant that is more easily insertable into an SI joint in its collapsed state than SI implants of the prior art.

It is another object of the present invention to provide an SI joint implant comprising anchor members configured to move apart laterally to expand the SI joint and/or fixate the SI joint in an expanded position.

### BRIEF DESCRIPTION OF DRAWINGS

Accompanying this written specification is a collection of drawings of exemplary embodiments of the present invention. One of ordinary skill in the art would appreciate that these are merely exemplary embodiments, and additional and alternative embodiments may exist and still be within the spirit of the invention as described herein.

FIG. 1 is a perspective view of an implant in a first, collapsed position in accordance with embodiments of the present invention.

FIG. 2 is a perspective view of an implant in a second, expanded position in accordance with embodiments of the present invention.

FIG. 3 is a perspective view of an anchor member in accordance with embodiments of the present invention.

FIG. 4 is a perspective view of an arm member in accordance with embodiments of the present invention.

FIG. 5 is a rear (posterior) view of a pelvis with an implant placed in an implanted position in accordance with embodiments of the present invention.

### DETAILED DESCRIPTION

In accordance with embodiments of the present invention, an implant for partial to full fixation of a sacroiliac (SI) joint may comprise a pair of anchor members connected together by an adjustable connector assembly. In some embodiments, the adjustable connector assembly may comprise a pair of arm members configured to pivot about one or more axes to move the implant from a first position to a second position, for example, from a partially or fully collapsed position to a partially or fully expanded position.

In accordance with embodiments of the present invention, the anchor members may be elongated components with anti-migration features running generally perpendicular to the long axis on the exterior of an anchor member. The anti-migration features may be teeth, threads, a roughened surface, or any other similar textured or roughened surface suitable for preventing migration of an implanted implant. The anchor members may be generally cylindrical, but could also be a conical, triangular, rectangular or any other similarly suitable shape for implantation. In some embodiments, a slot may run axially along an axis of the anchor. The slot may extend from the top (superior) side to the bottom (inferior) side of the anchor. A pivot point may also extend from the top (superior) side to the bottom (inferior) side of the anchor member. In some embodiments, the slot may be positioned in a location generally near the rear (posterior) end of the anchor member, and the pivot point may be positioned in a location generally near the front (anterior) of the anchor member. A posterior cavity may extend axially along the anchor from the rear (posterior) face to at least the slot feature. An elongate lateral opening or slot may extend axially along the side (medial) wall of the anchor member. The pair of anchor members may be oriented in such a way so that the lateral openings face each other.

In accordance with embodiments of the present invention, the anchor members may be connected together by an adjustable connector assembly. The adjustable connector assembly may be configured to adjust (e.g., decrease or increase) the distance between the anchor members and may comprise a pair of arm members in pivotal communication with one another. In accordance with embodiments of the present invention, the arm members may be able to pivot and move along various axis points, for example, about a series or set of one or more pivotable fasteners (or one or more pairs of pivotable fasteners), for example, posts, pins, screws, or any other similarly suitable pivotable fasteners. In some examples, portions of the arm members may be received inside the lateral openings of the anchor members. For example, a first arm member may have its first end received in a lateral opening of a first anchor member and a second end received in a lateral opening of a second anchor member, and a second arm member may have its first end received in the lateral opening of the second anchor member and its second end received in the lateral opening of the first anchor member.

In accordance with embodiments of the present invention, the arm members may comprise a central pivot point, rear (posterior) pivot point, and a front (anterior) pivot point. A rear (posterior) pin may extend through the slot of the anchor member and into the rear (posterior) pivot point of the arm member. The arm member may pivot about the rear (posterior) pin. Also, the rear (posterior) pin may slide within the guide slot of the anchor member. This may allow the rear (posterior) pin, and the arm member, to translate front to rear (e.g., anterior-posterior) along the central axis of the anchor member. A front (anterior) pin may extend through the pivot point of the anchor member and into the front (anterior) pivot point of the arm member. The arm member may then pivot about the front (anterior) pin. Similarly, a central pin may be received within the central pivot point of the arm members, allowing the arm members to pivot at the central pivot point with respect to each other.

In accordance with an exemplary usage scenario, an implant in accordance with embodiments of the present invention may be inserted into the sacral-iliac joint. A first anchor member may be implanted into a first bone, for example, the sacrum, while the second anchor member may be implanted into a second bone, for example, the ilium (e.g., contralateral of the SI Joint) with respect to the first anchor member. In some scenarios, during insertion, the pair of anchors may be relatively close to one another, with the posterior pins being positioned in a rear (e.g., posterior) position, for example, in a position farthest from the front (anterior) pins. In some embodiments, once the implant is positioned (e.g., seated) in the SI joint, the implant may be expanded. For example, the arm members may be pressed forward with a tool (e.g., an inserting or expanding tool) through the rear (posterior) cavity. In some examples, forcing a rear (posterior) portion of the arm members forward (e.g., sliding the arm members within the anchor member shafts) directs the arm members to pivot about the front (anterior) pin and the central pin. Such an action may move (e.g., force) the anchor members apart laterally, expanding the SI joint and/or fixating the SI joint in an expanded position. In some examples, the arm members may be secured (e.g., locked) in an expanded position. In some embodiments, one or more fasteners may be utilized to secure (e.g., lock) the arm members in an expanded position. The fasteners may be set screws, pins, nails or other similarly suitable fastening implements. For example, the posterior openings may be formed with threads or threaded portions configured to engage with threaded set screw(s) to secure the set screws to the assembly and maintain the arm members in their forward (e.g., expanded implant) position. In an illustrative example, a set screw may be configured to abut a posterior (rear) end of the arm members to hold the arm members in a second position wherein the implant is expanded.

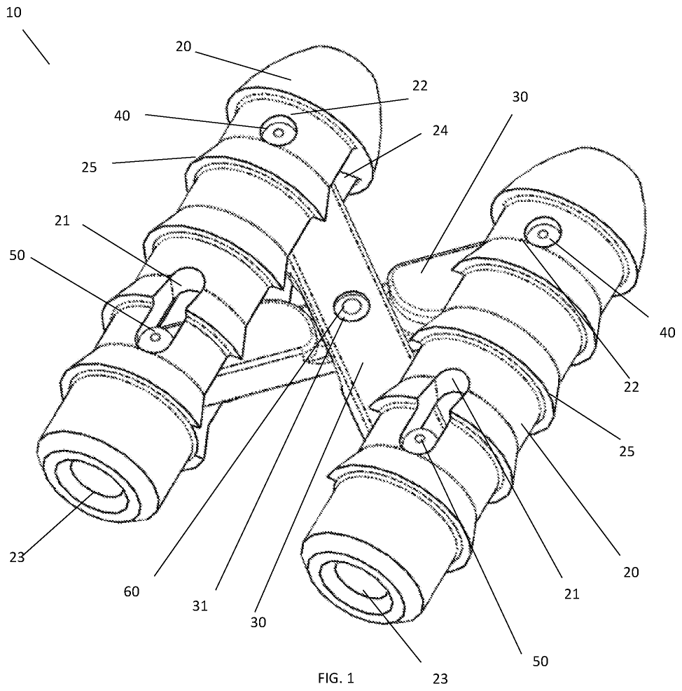

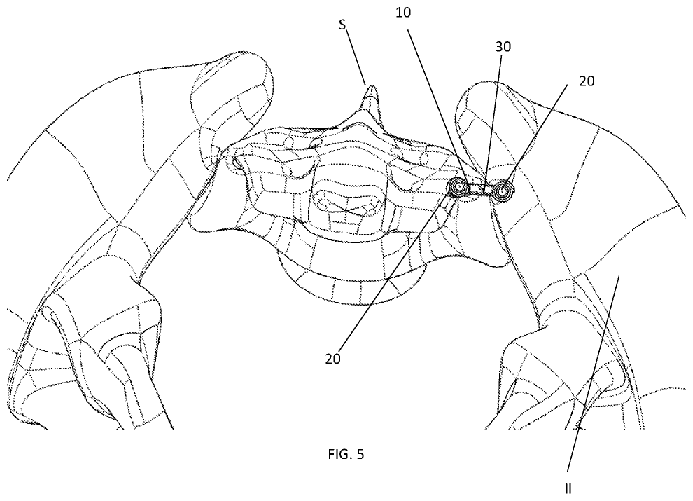

Turning now to the figures, FIGS. 1-5 generally depict an implant for SI joint fixation in accordance with embodiments of the present invention. As generally shown in FIGS. 1-5, an implant 10 for partial to full fixation of an SI joint may comprise a pair of anchor members 20 connected together by an adjustable connector assembly. In some embodiments, the adjustable connector assembly comprises a pair of pivotable arm members 20.

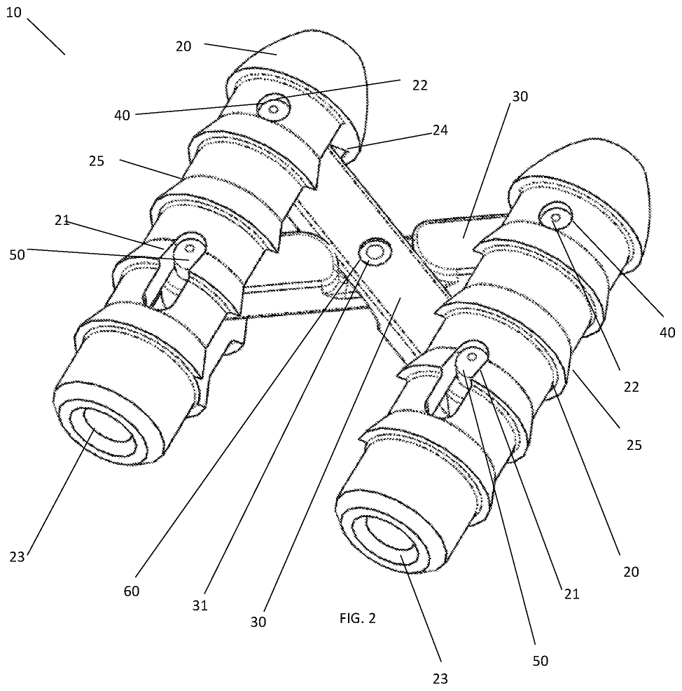

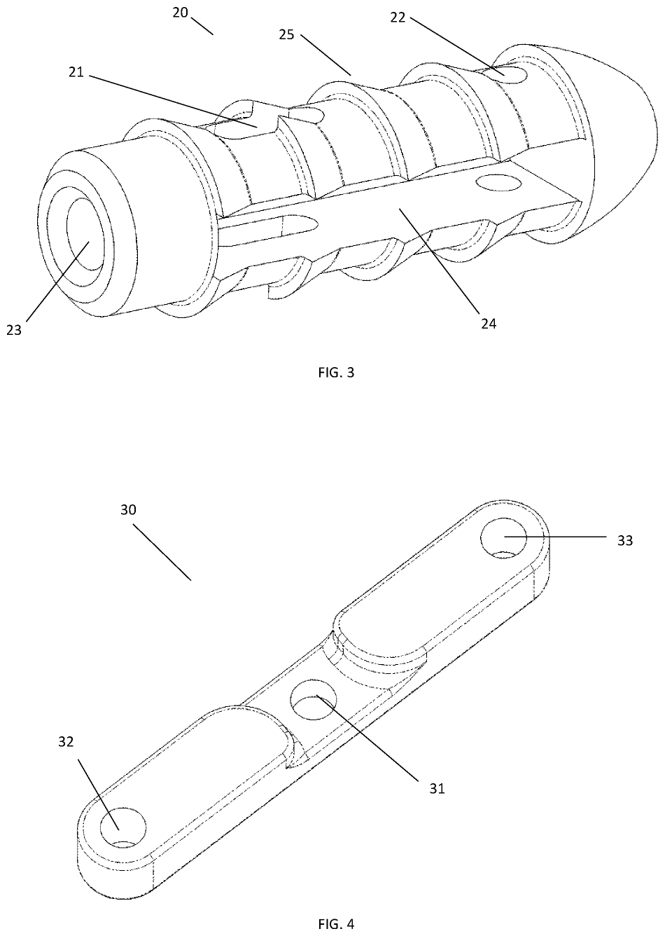

FIG. 1 depicts an embodiment of the implant of the present invention in an exemplary first, collapsed position, FIG. 2 depicts an embodiment of the implant of the present invention in an exemplary second, expanded position, and FIG. 3 depicts an exemplary anchor member of an exemplary implant of the present invention. As shown in FIGS. 1-3, the anchor members 20 of an implant 10 may be elongated components with anti-migration features 25 running generally perpendicular to the long axis on the exterior of an anchor member 20. The anti-migration features 25 may be teeth, threads, a roughened surface, or any other similar textured or roughened surface suitable for preventing migration of an implanted implant 10. The anchor members 20 may be generally cylindrical, but could also be a conical, triangular, rectangular or any other similarly suitable shape for implantation. In some embodiments, a slot 21 may run axially along an axis of the anchor 20. For example, the slot 21 may extend from the top (superior) side to the bottom (inferior) side of the anchor 20. A pivot point 22 may also extend from the top (superior) side to the bottom (inferior) side of the anchor member 20. In some embodiments, the slot 21 may be positioned in a location generally near the rear (posterior) end of the anchor member 20, and the pivot point 22 may be positioned in a location generally near the front (anterior) of the anchor member 20. A rear (posterior) cavity 23 may extend axially along the anchor 20 from the rear (posterior) face to at least the slot 21 feature. A side (lateral) opening 24 may extend axially along the side (e.g., medial) wall of the anchor member 20. The pair of anchor members 20 may be oriented in such a way so that the side (lateral) openings 24 face each other. As shown for example in FIGS. 1 and 2, the side (lateral) openings 24 may be configured to receive a portion of an arm member, for example, an end portion of an arm member. In some embodiments, each side (lateral) opening of each anchor member 20 may be configured to receive an end portion of each of the pair of arm members 30.

As further shown in FIGS. 1-2, in some embodiments, the anchor members 20 may be connected together by an adjustable connector assembly. The adjustable connector assembly may comprise a pair of arm members 30 in pivotal communication with one another. As shown in FIG. 4, the arm members 30 may comprise one or more axis points, for example, axis points 41, 51, and 61. In accordance with embodiments of the present invention, the arm members 30 may be able to pivot and move along pivot or axis points 31, 32, and 33, for example, about a series or set of one or more pins (or one or more pairs of pins) 40, 50, 60. End portions of the arm members 30 may be received inside the lateral openings 24 of the anchor members 20.

As further shown in FIG. 4, the arm members 30 may comprise a central pivot point 31, rear (posterior) pivot point 32, and a front (anterior) pivot point 33. A rear (posterior) pin 50 may extend through the slot 21 of the anchor member 20 and into the rear (posterior) pivot point 32 of the arm member 30. The arm 30 may pivot about the rear (posterior) pin 50. Also, the rear (posterior) pin 50 may slide within the guide slot 21 of the anchor member 20. This may allow the rear (posterior) pin 50, and the arm member 30 to translate front to rear (e.g., anterior-posterior) along the central axis of the anchor member 20. A front (anterior) pin 40 may extend through the pivot point 22 of the anchor member 20 and into the front (anterior) pivot point 33 of the arm member 30. The arm member 30 may then pivot about the front (anterior) pin 40. Similarly, a central pin 60 may be received within the central pivot point 31 of the arm members 30, allowing the arm members 30 to pivot with respect to each other.

In accordance with an exemplary usage scenario, an implant 10 in accordance with embodiments of the present invention may be inserted into the sacral-iliac joint. A first anchor member 20 may be implanted into the sacrum, while the second anchor member 20 may be implanted contralateral of the SI Joint into the ilium with respect to the first anchor member 20. In some scenarios, during insertion, the pair of anchors 20 may be relatively close to one another, with the rear (posterior) pins 50 being positioned in a rear (e.g., posterior) position, for example, in a position farthest from the front (anterior) pins 40. In some embodiments, once the implant 10 is positioned (e.g., seated) in the SI joint, the implant 10 may be expanded. For example, the arm members 30 may be pressed forward with a tool (e.g., an inserting or expanding tool) through the rear (posterior) cavity 23. In some examples, forcing a rear (posterior) portion of the arm members 30 forward directs the arm members 30 to pivot about the front (anterior) pin 40 and the central pin 60. Such an action may move (e.g., force) the anchor members 20 apart laterally, expanding the SI joint and/or fixating the SI joint in an expanded position.

In another exemplary usage scenario, a tool may be inserted into the interior area of the anchor members through their rear (posterior) openings 23 to support a transition of the implant 10 from a collapsed position (as shown for example in FIG. 1) to an expanded position (as shown for example in FIG. 2), for example, by pushing the second ends of the arm members 30 forward, directing the pins engaged thereto along a path permitted by the guide slots 21 of the anchor members 20, and causing the arm members 30 to pivot about their respective pivot axis to increase the distance (e.g., the lateral distance) between the anchor members 20. In some embodiments, the guide slots 21 may be grooves or any other similar indent or opening configured as a path or movement boundary for a post or pin to travel along. In some examples, the arm members 20 may be secured (e.g., locked) in an expanded position. For example, the rear (posterior) openings 23 may be formed with threads or threaded portions configured to engage with threaded set screw(s) to secure the set screws to the assembly and maintain the arm members 20 in their forward (e.g., expanded implant) position. In an illustrative example, a set screw may be configured to abut a posterior (rear) end of the arm members 20 to hold the arm members 20 in a second position wherein the implant 10 is expanded.

In the Summary above and in this Detailed Description, and the Claims below, and in the accompanying drawings, reference is made to particular features of various embodiments of the invention. It is to be understood that the disclosure of embodiments of the invention in this specification includes all possible combinations of such particular features. For example, where a particular feature is disclosed in the context of a particular aspect or embodiment of the invention, or a particular claim, that feature can also be used—to the extent possible—in combination with and/or in the context of other particular aspects and embodiments of the invention, and in the invention generally.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

In the present disclosure, various features may be described as being optional, for example, through the use of the verb “may;”, or, through the use of any of the phrases: “in some embodiments,” “in some implementations,” “in some designs,” “in various embodiments,” “in various implementations,”, “in various designs,” “in an illustrative example,” or “for example;” or, through the use of parentheses. For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be embodied in seven different ways, namely with just one of the three possible features, with any two of the three possible features or with all three of the three possible features. In various embodiments. elements described herein as coupled or connected may have an effectual relationship realizable by a direct connection or indirectly with one or more other intervening elements.

In the present disclosure, the term “any” may be understood as designating any number of the respective elements, i.e. as designating one, at least one, at least two, each or all of the respective elements. Similarly, the term “any” may be understood as designating any collection(s) of the respective elements, i.e. as designating one or more collections of the respective elements, a collection comprising one, at least one, at least two, each or all of the respective elements. The respective collections need not comprise the same number of elements.

While various embodiments of the present invention have been disclosed and described in detail herein, it will be apparent to those skilled in the art that various changes may be made to the configuration, operation and form of the invention without departing from the spirit and scope thereof. In particular, it is noted that the respective features of embodiments of the invention, even those disclosed solely in combination with other features of embodiments of the invention, may be combined in any configuration excepting those readily apparent to the person skilled in the art as nonsensical. Likewise, use of the singular and plural is solely for the sake of illustration and is not to be interpreted as limiting.

In the present disclosure, all embodiments where “comprising” is used may have as alternatives “consisting essentially of,” or “consisting of.” In the present disclosure, any method or apparatus embodiment may be devoid of one or more process steps or components. In the present disclosure, embodiments employing negative limitations are expressly disclosed and considered a part of this disclosure.

Certain terminology and derivations thereof may be used in the present disclosure for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “comprises” and grammatical equivalents thereof are used herein to mean that other components, ingredients, steps, among others, are optionally present. For example, an embodiment “comprising” (or “which comprises”) components A, B and C can consist of (i.e., contain only) components A, B and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most fastener %” means fastener % or less than fastener %. When, in this specification, a range is given as “(a first number) to (a second number)” or “(a first number)-(a second number),” this means a range whose limit is the second number. For example, 25 to 100 mm means a range whose lower limit is 25 mm and upper limit is 100 mm.

Many suitable methods and corresponding materials to make each of the individual parts of embodiment apparatus are known in the art. According to an embodiment of the present invention, one or more of the parts may be formed by machining, 3D printing (also known as “additive” manufacturing), CNC machined parts (also known as “subtractive” manufacturing), and injection molding, as will be apparent to a person of ordinary skill in the art. Metals, wood, thermoplastic and thermosetting polymers, resins and elastomers as may be described herein-above may be used. Many suitable materials are known and available and can be selected and mixed depending on desired strength and flexibility, preferred manufacturing method and particular use, as will be apparent to a person of ordinary skill in the art.

Any element in a claim herein that does not explicitly state “means for” performing a specified function, or “step for” performing a specific function, is not to be interpreted as a “means” or “step” clause as specified in 35 U.S.C. § 112 (f). Specifically, any use of “step of” in the claims herein is not intended to invoke the provisions of 35 U.S.C. § 112 (f).

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

1. An implant comprising:
a pair of anchor members, each anchor member comprising an elongate slot formed in a central portion of a side wall of the anchor member;
a pair of arm members operably connecting the anchor members to one another, each of the arm members pivotally intersecting with one another and having a first end pivotally received in a front portion of a first elongate slot of the two and a second end slidably received in a rear portion of a second elongate slot of the two; and
wherein the arm members pivot about a pivot axis disposed in their middle sections to expand the implant by increasing the distance between the anchor members.

2. The implant of claim 1, wherein the anchor members expand simultaneously.

3. The implant of claim 1, wherein expanding the anchor members supports distraction of pelvic bones.

4. The implant of claim 1, wherein a rear opening formed in each of the anchor members provides access to a rear portion of one of the arm members.

5. An expandable sacroiliac joint implant comprising:
a pair of anchor members, each anchor member comprising an elongate slot formed in a side wall of the anchor member, the elongate slot formed with a front arm engagement point and a rear arm engagement area;
a pair of arm members pivotally intersecting at a pivot axis, the arm members having a front end received in the elongate slot of one anchor member of the pair and pivotally engaged with the front arm engagement point of said anchor member and a rear end received in a rear portion of the elongate slot of an opposing anchor member of the pair and slidably engaged with the rear arm engagement area of said opposing anchor member; and
wherein the implant is configured to transition from a collapsed position to an expanded position when the second ends of the arm members are directed forward such that the arm members pivot about their respective pivot axes and increase the distance between the anchor members.

6. The expandable sacroiliac joint implant of claim 5, wherein a pair of openings are formed at the front arm engagement point on opposing top and bottom walls of each anchor member.

7. The expandable sacroiliac joint implant of claim 5, wherein a pair of guide slots are formed at the rear arm engagement area on opposing top and bottom walls of each anchor member.

8. The expandable sacroiliac joint implant of claim 7, wherein the rear end of each arm member is engaged with a pin configured to travel in the slots of one of the pair of anchor members.

9. The expandable sacroiliac joint implant of claim 5, wherein each anchor member is formed with a rounded conical head portion.

10. The expandable sacroiliac joint implant of claim 5, wherein the anchor members comprise threaded sections adapted to engage with bone to prevent migration of the implant.

11. An expandable implant for sacroiliac joint adjustment comprising:
a pair of anchor members, each anchor member comprising a rear opening providing access to an interior area of the anchor member, an elongate slot formed in a central portion of a side wall of the anchor member, and top and bottom walls, each of the top and bottom walls formed with a pin opening and a guide slots;
a pair of arm members pivotally intersecting at a pivot axis disposed on their respective middle sections, each arm member having a first end received in a front portion of a first elongate slot of the two and having a first pin pivotally engaged with the pin openings of one of the anchor members and a second end received in a rear portion of a second elongate slot of the two and having a second pin slidably engaged with the pin slots of one of the anchor members; and
wherein a tool is insertable into the interior area of the anchor members to support a transition of the implant from a collapsed position to an expanded position by directing the second ends of the arm members forward along a path permitted by the guide slots of the anchor members to pivot the arm members about their respective pivot axis and increase the distance between the anchor members.

12. The expandable implant for sacroiliac joint adjustment of claim 11, wherein the top wall pin opening opposes the bottom wall pin opening and the top wall guide slot opposes the bottom wall pin guide.

13. The expandable implant for sacroiliac joint adjustment of claim 11, wherein the arm members are each formed with a central groove wherein the pivot axis is disposed.

14. The expandable implant for sacroiliac joint adjustment of claim 11, wherein the anchor members comprise threads adapted to engage with bone to prevent migration of the implant.

15. The expandable implant for sacroiliac joint adjustment of claim 11, wherein the guide slots are configured as movement boundaries for the respective second pins of the arm members.

16. The expandable implant for sacroiliac joint adjustment of claim 11, wherein the guide slots regulate movement of the arm members relative to the anchor members.

17. The expandable implant for sacroiliac joint adjustment of claim 11, wherein in the deployed position, the arm members are configured to prevent the anchor members from collapsing back into the main body.
